(19) United States
(12) Patent Application Publication
(10) Pub. No.: US 2021/0212973 A1
(43) Pub. Date: Jul. 15, 2021

(54) CARDIOPROTECTIVE AMINO ACID FORMULATION

(71) Applicant: Almeda Labs LLC, Kansas City, MO (US)

(72) Inventors: Stacy Tucker, Kansas City, MO (US); Sushrutha Nagaraj, Kansas City, MO (US)

(21) Appl. No.: 17/263,677

(22) PCT Filed: Jul. 25, 2019
(86) PCT No.: PCT/US2019/043365

§ 371 Date: Jan. 27, 2021

Related U.S. Application Data
(60) Provisional application No. 62/712,035, filed on Jul. 30, 2018.

(51) Int. Cl. A61K 31/198; A61K 31/4172; A61K 31/401; A61K 9/00; A61K 9/48; A23L 33/175; A23L 33/00

(52) U.S. Cl. CPC A61K 31/198; A61K 31/4172; A61K 31/401; A23V 2002/00; A61K 9/4816; A23L 33/175; A23L 33/40; A61K 9/0056

(57) ABSTRACT

Amino acid formulations for cardiac vitality. The formulations comprise a mixture of a plurality of amino acids, wherein the mixture comprises at least one amino acid selected from the group consisting of serine, threonine, histidine, and/or proline, and optionally arginine Methods of enhancing smooth muscle cell vitality, stabilizing cardiac health, and decreasing oxidative stress and inflammation are also described.

### CROSS-REFERENCE TO RELATED APPLICATIONS

The present application claims the priority benefit of U.S. Provisional Patent Application Ser. No. 62/712,035, filed Jul. 30, 2018, entitled CARDIOPROTECTIVE AMINO ACID FORMULATIONS, incorporated by reference in its entirety herein.

### BACKGROUND OF THE INVENTION

### Field of the Invention

The present invention relates to synergistic amino acid formulations for enhancing cellular health and viability underlying cardiac health.

### Description of Related Art

Cardiovascular conditions and anxiety disorders all cause substantial morbidity to patients and costs to the healthcare system. A wide variety of research has demonstrated an association between negative psychological states and cardiovascular health. For example, anxiety, anxiety disorders, and depression are risk factors for cardiovascular events (e.g., arrhythmias, coronary artery disease, heart failure, heart attack, hypertension) as well as neurovascular disease, and implicated in overall heart health. Thus, the underlying causes of cardiovascular disease are complex and involve psychological, biological, and behavioral mechanisms. For example, research has shown that the heightened arousal associated with the spectrum of anxiety disorders (e.g., PTSD, panic disorders, obsessive compulsive disorders, generalized anxiety) is associated with an increased risk of hypertension, a pro-inflammatory state, and ultimately the development of coronary heart disease. These conditions have also been shown to each involve impaired vascular smooth muscle (VSM) function. More recent research has shown that anxiety potentially increases vascular events through worsening of vascular function in atherosclerotic disease and other cardiovascular conditions.

Thus, although psychological in nature, the physiological impact of anxiety cannot be denied, including: tachycardia (which in serious cases can interfere with normal heart function and even result in sudden cardiac arrest), increased blood pressure (which can lead to coronary heart disease, weakening of the heart muscle, and heart failure), decreased heart rate variability, as well as dizziness, chest pains, stomach discomfort, and shortness of breath.

Most prior art and medicinal approaches have been aimed at treating the symptoms, rather than targeting the underlying cause, either psychologically or nutritionally. For example, the primary emphasis should be on enhancing the cellular health and viability underlying cardiac health, so that the individual can better respond to situations of trauma and stress, whether physical or psychological in nature.

### SUMMARY OF THE INVENTION

Described herein are amino acid formulations for cardiac health. The formulations consist essentially of a mixture of a plurality of amino acids, wherein the amino acid mixture comprises at least two amino acids selected from the group consisting of serine, threonine, histidine, and/or proline. Preferably, the mixture comprises at least 3, and more preferably all 4 of the amino acids. The mixture may further optionally comprise arginine.

The invention is also concerned with nutritional and/or medicinal food products comprising an amino acid formulations, such as snack or meal replacement bars, powders, smoothies, shakes, juices, and gels having the amino acid mixture blended therein.

Methods of enhancing smooth muscle cell vitality are also described. The methods include contacting a smooth muscle cell with a therapeutically effective amount of an amino acid formulation according to the various embodiments, such that the smooth muscle cell exhibits stabilized or reduced contraction in response to a stressor. These methods include therapeutically or prophylactically administering a formulation to a subject in need thereof for a therapeutically effective amount of time. Such therapeutic or prophylactic treatments can be used to reduce inflammation and oxidative stress and promote cardiac vitality in the individual treated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph comparing human vascular smooth muscle cells' calcium fluorescence in response to the acetylcholine in the presence of the inventive amino acid formulation or the control.

FIG. 2 shows images of human vascular smooth muscle cells incubated with a negative control amino acid blend or the inventive amino acid formulation and staining of live (no stain) and the dead cells (with red nuclei or cytoplasm).

FIG. 3A is a graph showing calcium fluorescence in human vascular smooth muscle cells over time incubated with the inventive amino acid formulation in the presence of acetylcholine.

FIG. 3B is a graph showing calcium fluorescence in human vascular smooth muscle cells over time incubated with the negative control blend in the presence of acetylcholine.

FIG. 3C is a graph of the combined data from FIG. 3A and FIG. 3B.

FIG. 4 is a bar graph of the peak calcium transient effect of each of the negative control formulation and the inventive formulation on human vascular smooth muscle cells.

### DETAILED DESCRIPTION

The present invention is concerned with amino acid formulations for cardiac health, and specifically cardioprotective amino acid formulations for improving smooth muscle cell vitality. The amino acid formulations reduce oxidative stress and inflammatory responses. The individual amino acids are combined in synergistic blends according to the embodiments of the invention.

In one or more embodiments, an amino acid formulation is provided which comprises a mixture of amino acids, wherein the amino acids are selected from at least one of serine, threonine, histidine and/or proline. In one or more embodiments, amino acid formulations according to the invention further comprise at least arginine. In one or more embodiments, amino acid formulations consist of a mixture of amino acids, wherein the amino acids are selected from one or more of arginine, serine, threonine, histidine, and/or proline. The mixture of amino acids comprises at least 4 different amino acids, even more preferably, at least 5 different amino acids, and most preferably 5 different amino acids blended together in a unit dosage formulation.

In one or more embodiments, a first amino acid formulation is provided, which consists essentially (or even consists) of a mixture of serine, threonine, histidine, proline, and optionally arginine. As used herein, the phrase “consisting essentially” or “consists essentially” of means that the formulations are preferably limited to the specified ingredients (amino acids), but allow for the inclusion of minor impurities, additives, fillers, etc. that do not materially affect the basic characteristics of the formulation.

Regardless of the embodiment, the amino acids are preferably blended in an about 1:1 weight ratio or about 1:1.2 weight ratio for some amino acids (e.g., those provided in salt form, such as lysine to ensure the amount of available amino acid is about 1:1). Exemplary formulations are essentially free of any other additives and impurities, it being appreciated that minor amounts of impurities may be present due to amino acid manufacturing processes. In any case, pharmaceutical grade amino acids (i.e., 99% pure, discrete amino acids not conjugated to other proteins) are preferably used. Unless otherwise indicated, references herein to “amino acids” includes functionalized forms thereof (e.g., esterified or acylated) and/or salts thereof (e.g., HCl, acetates, sulfates, glutamates, etc.). As noted above, it will be appreciated that when functionalized or salt forms are used in a blend, the amount used may be adjusted (increased) to ensure that the total amount of available amino acid (active) is maintained. It will also be appreciated that amino acids used in the invention are L-form amino acids, whether or not expressly specified in referring to the particular amino acid.

In one or more embodiments, the mixture is substantially free of and comprises less than 5% by weight, preferably less than 1% by weight, an even more preferably less than 0.5% by weight of one or more (and in some cases all of) amino acids selected from the group consisting of: alanine, aspartic acid, glutamine, glycine, leucine, methionine, phenylalanine, tryptophan, tyrosine, cysteine, and valine. In other words, such amino acids are not intentionally added, and are preferably excluded in the inventive formulations.

Preferably, the amino acid formulation is provided as a unit dosage form, particularly suitable for oral administration. The unit dosage form can be from about 400-600 mg, preferably from about 500-600 mg, more preferably from about 500-550 mg, and even more preferably about 500-520 mg (where the term “about” refers to +/−5mg from the indicated amount). In one or more embodiments, the formulation is suitable for a total daily dosage form of from about 0.5 grams to about 2 grams per day, preferably from about 0.6 grams to about 1.5 grams per day, and more preferably from about from about 0.6 grams to about 1.2 grams per day (where the term “about” refers to +/−0.2 grams from the indicated amount). In certain embodiments, the amino acid formulation is provided in an oral supplement, which can be selected from the group consisting of pill, tablet, capsule, liquid solution, gel cap, and the like. In certain embodiments, the amino acids are in powder form and encapsulated in a vegetable capsule. Extended release capsules may also be used.

It is also contemplated that the amino acid formulations can be provided as part of a nutritional or medicinal food product, such as snack or meal replacement bars, powders, smoothies, shakes, juices, gels, and the like. Regardless, the low-dosage combination of the four or five amino acid blends produces a rather unique synergistic modulation which translates into greater cardiac vitality.

In one or more embodiments, a therapeutically effective amount of the amino acid formulation is administered to a subject in need thereof for a therapeutically effective amount of time. Such individuals include those suffering from trauma, anxiety, or stress affecting cardiovascular vitality and/or function generalized anxiety disorder, post-traumatic stress disorder, obsessive compulsive disorder, panic attack, and the like. Subjects for treatment with the inventive formulation also include individuals with risk factors for cardiovascular and neurovascular disease to provide a cardioprotective effect against future stress or trauma. Thus, the formulations may be used prophylactically before the individual is exposed to or exhibiting symptoms of stress or anticipated trauma (whether physical or emotional). Likewise, the formulations may be used therapeutically after an individual has been exposed and/or is exhibiting symptoms of stress or trauma. As used herein, the term “therapeutically effective” refers to the amount and/or time period that will elicit the biological or medical response of a tissue, system, animal, or human that is being sought by a researcher or clinician, and in particular elicit some desired therapeutic effect. For example, in one or more embodiments, therapeutically effective amounts and time periods are those that enhance, improve, maintain vascular or cardiac cell functioning and vitality (e.g., as exemplified by stabilized or reduced smooth muscle cell contractions in response to stressors).

One of skill in the art recognizes that an amount or time period may be considered “therapeutically effective” even if the condition is not totally eradicated but improved partially. Exemplary dosages range from about 0.6 grams to about 1.2 grams of the formulation over a 24-hr period. Dosages can be repeated daily for a period of about 20 to about 40 weeks, or taken daily on an ongoing basis as needed.

The amino acid formulation reduces oxidative stress, decreases inflammation, and increases vascular vitality, and particularly vitality and function of smooth muscle cells of the vascular system. More particularly, the amino acid formulation reduces or stabilizes smooth muscle cell contractions in response to stress, trauma, and/or anxiety that manifests physiologically in the subject. For example, the formulation can help mitigate the cascade of hormones and neurotransmitters that occur in response to stress, trauma, and/or anxiety by reducing the cardiovascular or neurovascular response to such stress, trauma, and/or anxiety (e.g., through stabilization of smooth muscle cells function/contraction).

It will be appreciated that therapeutic and prophylactic methods and formulations described herein are applicable to humans as well as any suitable animal, including, without limitation, dogs, cats, and other pets, as well as, rodents, primates, horses, cattle, pigs, etc. The methods can be also applied for clinical research and/or study. Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Introduction

In this work, the function of vascular smooth muscle cells was analyzed by studying acetylcholine-induced calcium signaling and contraction in human smooth muscle cells incubated in the presence of the inventive amino acid formulation and controls. After long-term exposure to the formulation or control, the cells were loaded with calcium-sensitive fluorophore (calcium orange). Cells were subsequently placed on a laser scanning confocal microscope and exposed acutely to acetylcholine, which induces a rapid calcium transient in the cells (via an ion channel), which in turn induces contraction of the cells. In these studies, cells pre-incubated with the inventive amino acid formulation had a statistically lower calcium peak compared to the controls. Cells pre-incubated with the inventive formulation also had improved viability with a lower percentage of dead cells in each sample. Cells in both groups demonstrated normal smooth muscle cell morphology and no differences in growth rates.

### Example 1

Human vascular smooth muscle cells were removed from storage vial and expanded in culture with DMDM media at 37° C. and 5% CO₂. Cells were seeded at a density of 2500 cells/cm² in T25 flasks. Growth media was changed every 2-3 days, and cells were allowed to expand for 2 weeks. When enough cells were present, the cells were loaded into 96-well microplates and allowed to grow for another 3-5 days to reach confluency.

The amino acid formulation was prepared from the following components:

TABLE 1.1
Smooth Muscle Formulation

| Component | Weight (g) |
|---|---|
| L-Arginine | 4.9989 |
| L-Serine | 4.9998 |
| L-Threonine | 5.0011 |
| L-Histidine | 4.9994 |
| L-Proline | 5.0004 |

In order to keep the ratio of the components identical, this blend was used for all studies. The control cells were incubated without the amino acid formulation.

Rather than plating cells in a 96-well plate for testing, as described above, cells were plated in small petri dishes, with each well containing a glass coverslip. Cells attached to the coverslip within 7 days and were ready for testing. On the day of testing, cells were incubated with the calcium-sensing fluorophore, Calcium Orange, at a concentration of 5 μM for 30 minutes. Subsequently, a coverslip was removed from the petri dish and placed in the Autofluor chamber with 150 μL of fresh DMEM on the confocal microscope. The confocal settings (in arbitrary units) were: PMT intensity 570, gain 32, offset 14% with a collection frequency of every 5 seconds. The basal calcium fluorescence measurement was collected for 30-60 seconds, prior to the application of μM acetylcholine. Acetylcholine was while continuously monitoring the emission added while monitoring fluorescence emission.

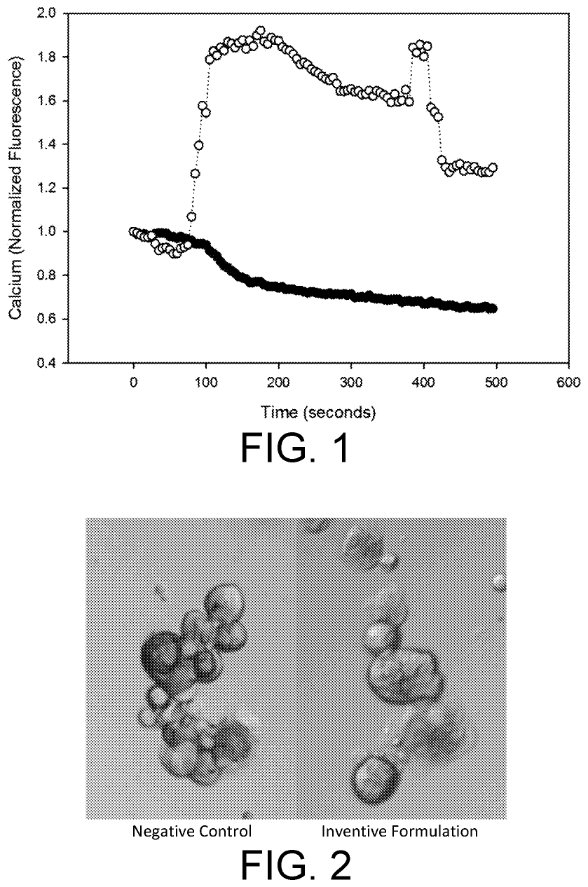

All of the control cells showed some type of acute response to the acetylcholine, although cell-to-cell variability was high. In contrast, however, the cells incubated with the inventive amino acid formulation there was no increase in calcium fluorescence in response to the acetylcholine, and no calcium transient noted. In one case, the calcium fluorescence actually declined. A comparison can be seen in FIG. 1, where the control is indicated by the open circles, and the amino acid formulation is the closed circles.

Control cells responded to the acetylcholine exposure with a calcium transient, although the magnitude, shape, and timing of the transients was variable. In cells pre-exposed to the amino acid formulation, the acetylcholine-induced transients were blocked.

### Example 2

Human vascular smooth muscle cells were removed from storage vial and expanded in culture with DMDM media at 37° C. and 5% CO₂. Cells were seeded at a density of 2500 cells/cm² in T25 flasks. Growth media was changed every 2-3 days, and cells were allowed to expand for 4 weeks.

The amino acid formulation was prepared from the following components.

TABLE 2.1
Smooth Muscle Formulation

| Component | Weight (g) |
|---|---|
| L-Arginine | 1.0018 |
| L-Serine | 0.9998 |
| L-Threonine | 1.0042 |
| L-Histidine | 1.0023 |
| L-Proline | 1.0112 |

A negative control formulation was prepared for testing using a combination of amino acids commonly used as supplements to demonstrate the synergistic behavior of the particular amino acids selected for the inventive formulation.

TABLE 2.2
Negative Control Blend

| Component | Weight (g) |
|---|---|
| L-Phenylalanine | 1.0019 |
| L-Leucine | 1.0024 |
| L-Arginine | 1.0140 |
| L-Tyrosine | 1.0041 |
| L-Cysteine | 1.0136 |

Each blend was diluted in DMEM for a final concentration of a working stock solution of 2 mg/mL. In order to keep the ratio of the components identical, these blends were used for all studies.

After cells had reached 60% confluency in the T25 flasks, either the inventive formulation or the negative control blend were added to the flask media for a final concentration of 1 mg/mL. Cells were bathed in the two Test Articles for 4 days prior to testing. On the fourth day, the cells were removed from the flasks and either dispersed into 96 well plates, or onto round cover slips in 6-well plates. Cells were allowed to settle to the base of the plates/coverslip for 3 hours.

Fresh acetylcholine was prepared from a stock solution of 1 mM acetylcholine in DPBS. Fresh Calcium Orange was prepared by adding DMSO to a single vial of Calcium Orange, and diluting in DPBS for a final concentration of 10 μM.

On the 4th day of exposure to either the inventive formulation or the negative control blend, images of cells in the flasks were obtained using a Ziess Axio Inverted microscope (Vert.A1) with Jenoptik software (ProgRes). There were no obvious differences in cell density or morphology from the images. Unstained cells are difficult to image using phase contrast, but both groups presented with cells that appeared healthy and under proliferative stages. There appeared to have been more pronounced nuclei in the cell in the inventive formulation, but this was a qualitative and not quantitative observation.

Cell viability was measured, after cells were removed from the flasks and plated into 96-well plates. Propidium iodide (1 μM final concentration) was added to each well from a stock solution, and allowed 30 minutes to incubate. Images were captured on a Biotek Cytation 5 plate reader. With reference to FIG. 2, images were analyzed by counting the live (no stain) cells and the dead cells (with red nuclei or cytoplasm), and reported as percent viable (alive). The red nuclear stain indicated dead cells. FIG. 2 shows images of cells stained with PI, which first selectively stains the DNA within the nucleus of cells with compromised cell membranes, indicative of dead cells. Later, as the cells enter a more advanced stage of apoptosis, the DNA will spread throughout the cytoplasm and the red stain will incorporate the entire cellular structure. On the left panel, one can see examples of strictly nuclear staining (small punctate staining) and more advanced cell death demonstrated by the larger red staining in the single cell in the bottom of the image.

After analyzing over 625 cells, it was determined that there was a difference in viability between the 2 groups with 19.5% dead cells in the Negative Control group and only 13.0% dead cells in the inventive formulation group.

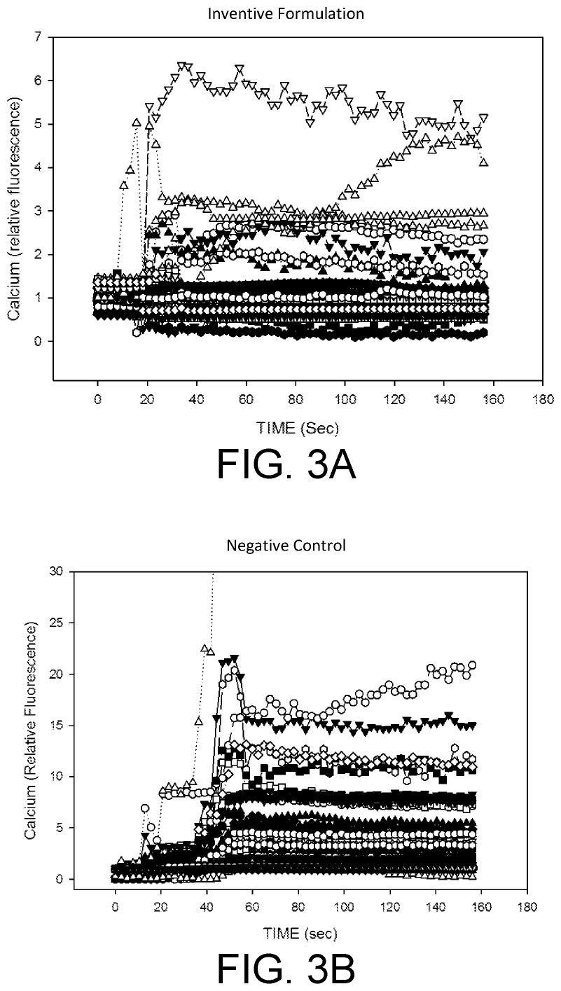

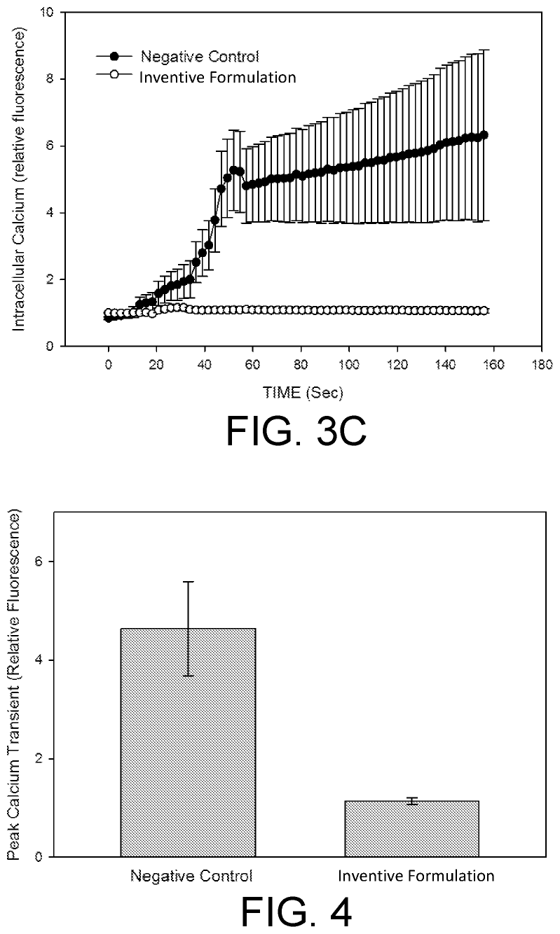

On the day of testing, cells were incubated with the calcium-sensing fluorophore, Calcium Orange, at a concentration of 5 μM for 30 minutes. Subsequently, the coverslip with cells attached was removed from the petri dish and placed in the Autofluor chamber with 150 μL of fresh DMEM on the confocal microscope. The confocal settings (in arbitrary units) were: PMT intensity 494, gain 2.5, offset 20% with a collection frequency of every 5 seconds. The basal calcium fluorescence measurement was collected for 20-30 seconds, prior to the application of acetylcholine (final concentration 100 μM). Acetylcholine was added while continuously monitoring the emission fluorescence. Overlaid graphs from the acetylcholine transients are shown in FIGS. 3A and 3B.

Importantly, there is great variability between cells. Within the inventive formulation group (FIG. 3A) there were some cells that showed a slight response to the acetylcholine challenge, while most of the cells did not. Alternatively, in the Negative control group (FIG. 3B), the majority of cells demonstrated some sort of response, although the magnitude of the responses varied greatly, even within the same experiment. Due to the high cell-to-cell variability, a large number of cells had to be analyzed in order to obtain a clear trend. 62 individual cells were analyzed from the negative control group. 98 individual cells were analyzed from the inventive formulation group.

Notably, while the 2 graphs appear to show partial responses in both groups, it is important to note that the Y axis on the 2 figures is different. The Y axis for the Negative Control group is nearly 5 times larger than the Y axis for the inventive formulation. Even then, one of the responders in the Negative Control group was so large that the upper peak of the transient is not shown in the graph. The data shows that the cells of the Negative Control group had a statistically greater response to the acetylcholine challenge than the inventive formulation group.

The differences between the 2 groups can best be seen with aggregated data, as shown in FIG. 3C. The panel graphs the average+standard error, calcium-excitation fluorescence at each time point. Overall, there was only a small response to acetylcholine in the inventive formulation group (SMC Blend), but in the Negative Control group there was a substantial response that remained high for the 2.5 minutes of monitoring. The large error bars in the Negative Control group that increased over time represent the tendency of some cells to return to basal calcium concentrations while other cells remained high. Thus, variation increased over time.

Another important calculation is to determine the peak of the calcium transient, no matter the time when it occurred, and compare the average peak values between the groups. The peak calcium transient was defined as the greatest value after the addition of acetylcholine. FIG. 4 shows the differences in the peak calcium transient, which was statistically greater (p<0.001) in the Negative Control group.

The acetylcholine-induced calcium transients were significantly greater in the Negative Control Blend compared to the inventive formulation when analyzed as both the calcium concentration over time and as the peak calcium transient. Cells growing in both media showed normal smooth muscle cell morphology with elongated cytoplasm, and able to shorten when unattached. Neither media altered growth rates. Finally, there was an improvement in viability with fewer dead cells in the inventive formulation group.

1. An amino acid formulation for cardiac health consisting essentially of a mixture of a plurality of amino acids, wherein the amino acid mixture comprises at least two amino acids selected from the group consisting of serine, threonine, histidine, and/or proline.

2. The amino acid formulation of claim 1, wherein said mixture further comprises arginine.

3. The amino acid formulation of claim 1, wherein said mixture comprises at least three of said amino acids.

4. The amino acid formulation of claim 1, wherein said mixture comprises all 4 of said amino acids.

5. The amino acid formulation of claim 4, further comprising arginine.

6. The amino acid formulation of claim 4, wherein said amino acids are blended together in unit dosage form.

7. The amino acid formulation of claim 6, wherein said unit dosage form comprises from about 400 to about 600 mg of said mixture.

8. The amino acid formulation of claim 7, wherein said unit dosage form is a pill, tablet, capsule, liquid solution, or gel cap.

9. The amino acid formulation of claim 4, wherein said amino acids are each blended in an about 1:1 weight ratio in said mixture.

10. The amino acid formulation of claim 4, wherein said mixture consists essentially of serine, threonine, histidine, proline, and optionally arginine.

11. The amino acid formulation of claim 1, wherein said mixture is substantially free of one or more amino acids selected from the group consisting of: alanine, aspartic acid, glutamine, glycine, leucine, methionine, phenylalanine, tryptophan, tyrosine, cysteine, and valine.

12. A nutritional or medicinal food product comprising an amino acid formulation according to claim 1.

13. The nutritional or medicinal food product of claim 12, selected from the group consisting of snack or meal replacement bars, powders, smoothies, shakes, juices, and gels having said amino acid mixture blended therein.

14. A method of enhancing smooth muscle cell vitality, said method comprising contacting a smooth muscle cell with a therapeutically effective amount of an amino acid formulation according to claim 1.

15. The method of claim 14, wherein said smooth muscle cell has stabilized or reduced contraction in response to a stressor after said contacting.

16. The method of claim 14, wherein said amino acid formulation is administered to a subject in need thereof for a therapeutically effective amount of time.

17. The method of claim 16, wherein said amino acid formulation is administered to said subject prophylactically.

18. The method of claim 16, wherein said subject exhibits stabilized or reduced smooth muscle cell contractions in response to stressors.

19. The method of claim 16, wherein said subject exhibits reduced inflammation and oxidative stress after said administering.

20. The method of claim 16, wherein said amino acid formulation is administered daily, twice per day as a unit dosage form.
